(19) United States
(12) Patent Application Publication
(10) Pub. No.: US 2009/0149651 A1
(43) Pub. Date: Jun. 11, 2009

(54) PROCESSES AND INTERMEDIATES FOR PRODUCING AMINOBENZIMIDAZOLE UREAS

(76) Inventors: Raymond Forslund, Natick, MA (US); Derek Magdziak, Medford, MA (US); Yong Dong, Wayland, MA (US); Gerald Tanoury, Hudson, MA (US)

Correspondence Address: VERTEX PHARMACEUTICALS INC., 130 WAVERLY STREET, CAMBRIDGE, MA, 02139-4242, US

(21) Appl. No.: 12/265,893

(22) Filed: Nov. 6, 2008

Related U.S. Application Data
(60) Provisional application No. 60/986,141, filed on Nov. 7, 2007.

(51) Int. Cl. C07D 401/14

(52) U.S. Cl. 544/333

(57) ABSTRACT

The present invention relates to processes and intermediates for the preparation of compounds useful as inhibitors of bacterial gyrase and Topoisomerase IV (Topo IV).

### CROSS-REFERENCE TO RELATED APPLICATIONS

The present application claims the benefit under 35 U.S.C. § 119 of U.S. provisional application Ser. No. 60/986,141, titled “PROCESSES AND INTERMEDIATES FOR PRODUCING AMINOBENZIMIDAZOLE UREAS ” filed Nov. 7, 2007, the entire contents of which is hereby incorporated by reference.

### TECHNICAL FIELD OF THE INVENTION

The present invention relates to processes and intermediates for the preparation of compounds useful as inhibitors of bacterial gyrase and Topoisomerase IV (Topo IV).

### BACKGROUND OF THE INVENTION

Bacterial resistance to antibiotics has long been recognized, and it is today considered to be a serious worldwide health problem. As a result of resistance, some bacterial infections are either difficult to treat with antibiotics or even untreatable.

Gyrase is one of the topoisomerases, a group of enzymes, which catalyze the interconversion of topological isomers of DNA (see generally, Komberg and Baker, DNA Replication, 2d Ed., Chapter 12, 1992, W. H. Freeman and Co.; Drlica, Molecular Microbiology, 1992, 6, 425; Drlica and Zhao, Microbiology and Molecular Biology Reviews, 1997, 61, 377). Gyrase itself controls DNA supercoiling and relieves topological stress that occurs when the DNA strands of a parental duplex are untwisted during the replication process. Gyrase also catalyzes the conversion of relaxed, closed circular duplex DNA to a negatively superhelical form, which is more favorable for recombination. The mechanism of the supercoiling reaction involves the wrapping of gyrase around a region of the DNA, double strand breaking in that region, passing a second region of the DNA through the break, and rejoining the broken strands. Such a cleavage mechanism is characteristic of a type II topoisomerase. The supercoiling reaction is driven by the binding of ATP to gyrase. The ATP is then hydrolyzed during the reaction. This ATP binding and subsequent hydrolysis cause conformational changes in the DNA-bound gyrase that are necessary for its activity. It has also been found that the level of DNA supercoiling (or relaxation) is dependent on the ATP/ADP ratio. In the absence of ATP, gyrase is only capable of relaxing supercoiled DNA.

Bacterial DNA gyrase is a 400 kilodalton protein tetramer consisting of two A (GyrA) and two B subunits (GyrB). Binding and cleavage of the DNA is associated with GyrA, whereas ATP is bound and hydrolyzed by the GyrB protein. GyrB consists of an amino-terminal domain, which has the ATPase activity, and a carboxy-terminal domain, which interacts with GyrA and DNA. By contrast, eukaryotic type II topoisomerases are homodimers that can relax negative and positive supercoils, but cannot introduce negative supercoils. Ideally, an antibiotic based on the inhibition of bacterial DNA gyrase would be selective for this enzyme and be relatively inactive against the eukaryotic type II topoisomerases.

Replication fork movement along circular DNA can generate topological changes both ahead of the replication complex as well as behind in the already replicated regions (Champoux, J. J., Ann. Rev. Biochem., 2001, 70, 369-413). While DNA gyrase can introduce negative supercoils to compensate for the topological stresses ahead of the replication fork, some overwinding can diffuse back into the already replicated region of DNA resulting in precatenanes. If not removed, the presence of the precatenanes can result in interlinked (catenated) daughter molecules at the end of replication. TopoIV is responsible for separating the catenated daughter plasmids as well as removal of precatenanes formed during replication ultimately allowing for segregation of the daughter molecules into daughter cells. Topo IV is composed of two ParC and 2 parE subunits as a C2E2 tetramer (where the C and E monomers are homologous to the A and B monomers of gyrase, respectively) that requires ATP hydrolysis (at the N-terminus of the E subunit) to reset the enzyme to re-enter the catalytic cycle. Topo IV is highly conserved among bacteria and is essential for bacterial replication (Drlica and Zhao, Microbiol. Mol. Biol. Rev., 1997, 61, 377).

Agents that can effectively inhibit multiple essential targets can result in an expanded spectrum of potencies, improved antibacterial potencies, improved potency against single target mutants, and/or lower spontaneous rates of resistance.

As bacterial resistance to antibiotics has become an important public health problem, there is a continuing need to develop newer and more potent antibiotics. More particularly, there is a need for antibiotics that represent a new class of compounds not previously used to treat bacterial infection. Such compounds would be particularly useful in treating nosocomial infections in hospitals where the formation and transmission of resistant bacteria are becoming increasingly prevalent.

Compounds described as gyrase and Topo IV inhibitors useful in the treatment of bacterial infections are disclosed in WO 02/060879, WO 05/0122292, US2005/0038247, US2006/0122196 and WO 07/056330. Also disclosed in these publications are processes and intermediates for the preparation of these compounds. There remains however, a need for economical processes for the preparation of these compounds.

### SUMMARY OF THE INVENTION

As described herein, one aspect of the present invention provides processes for preparing gyrase and Topo IV inhibitors useful in the treatment of bacterial infections. Such compounds include 1-ethyl-3-(5-(5-fluoropyridin-3-yl)-7-(pyrimidin-2-yl)-1H-benzo[d]imidazol-2-yl)urea (Compound 1) having the structure below:

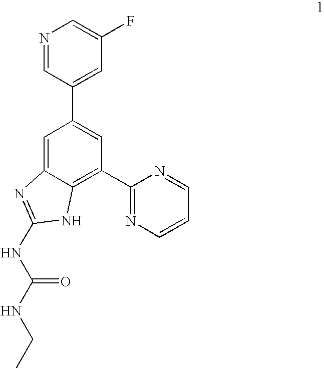

In another aspect, the present invention provides compounds useful as intermediates in the processes of the present invention.

### DETAILED DESCRIPTION OF THE INVENTION

In one aspect, the present invention relates to a process for preparing a compound of formula 1:

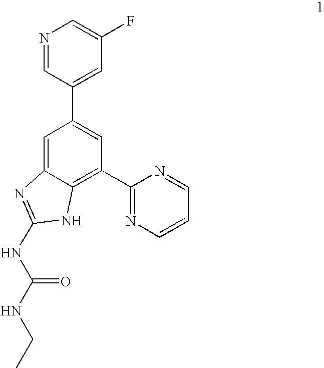

comprising:

i) providing 3-fluoro-5-(4,4,5,5-tetramethyl-1,3,2-dioxaborolan-2-yl)pyridine (formula 2) and 4-bromo-2-nitro-6-(pyrimidin-2-yl)benzenamine (formula 3),

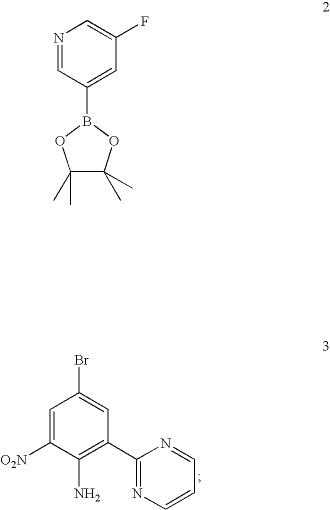

ii) cross coupling the compound of formula 2 and the compound of formula 3 in a biphasic mixture comprising water, an organic solvent, a base and a transition-metal catalyst to produce 4-(5-fluoropyridin-3-yl)-2-nitro-6-(pyrimidin-2-yl)benzenamine of formula 4,

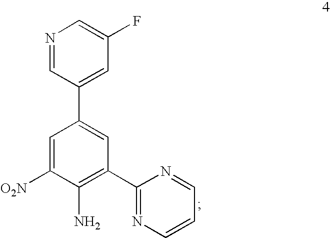

iii) reducing the compound of formula 4 to produce 5-(5-fluoropyridin-3-yl)-3-(pyrimidin-2-yl)benzene-1,2-diamine of formula 5,

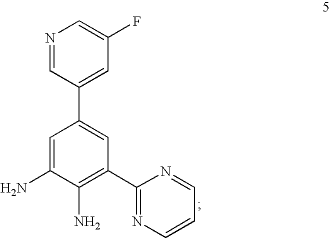

iv) reacting the compound of formula 5 with the compound of formula 6,

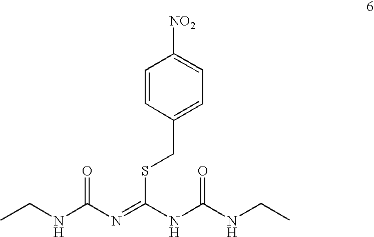

in a biphasic mixture comprising buffered water and an organic solvent to produce the compound of formula 1.

In another aspect, the present invention provides a process for preparing a compound of formula I:

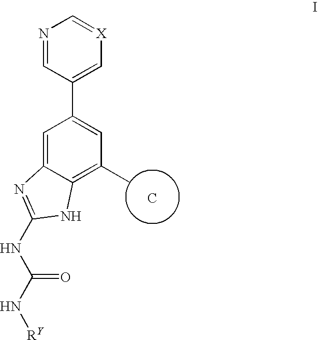

comprising:

ib) reacting a compound of formula 4b,

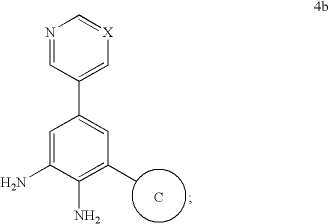

wherein,
Ring C is a 6-membered heteroaryl ring having 1-2 nitrogens, wherein:
Ring C is substituted with 1-3 R¹ groups;
   each R¹ is independently selected from OR² or halogen; and
   R² is C₁₋₄ aliphatic; or
Ring C is an unsubstituted 2-pyrimidine ring;
   X is nitrogen, CH, or CF;:
   with a compound of formula 6b,

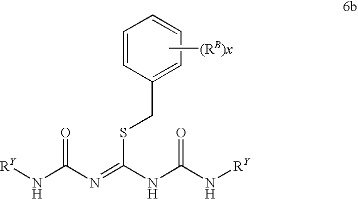

wherein,

x is 0-5;

R^{B} is selected from —R^{J}, —OR^{J}, —N(R^{J})₂, —NO₂, halogen, —CN, —C₁₋₄haloalkyl, —C₁₋₄haloalkoxy, —C(O)N(R^{J})₂, —NR^{J}C(O)R^{J}, —SOR^{J}, SO₂R^{J}, —SO₂N(R^{J})₂, —NR^{J}SO₂R^{J}, —COR^{J}, —NR^{J}SO₂N(R^{J})₂, —COCOR^{J}; wherein

R^{J} is hydrogen or unsubstituted C₁₋₆ aliphatic; and

each R^{Y} is C₁₋₆ aliphatic;

in a biphasic mixture comprising buffered water and an organic solvent to produce the compound of formula I.

In yet another aspect, the present invention also provides a process for purifying a compound of formula 1,

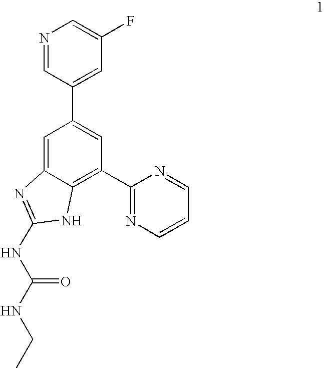

comprising:

a) slurrying a preparation comprising a compound of formula 1 in an organic solvent, water and acid to obtain a suspension of the compound of formula 1,

b) heating the suspension to obtain a homogeneous solution of compound 1,

c) filtering the homogeneous solution of compound 1,

d) cooling the solution to obtain a salt of compound 1 in a solid form.

In another aspect, the present invention also provides a process for preparing a compound of formula 6b,

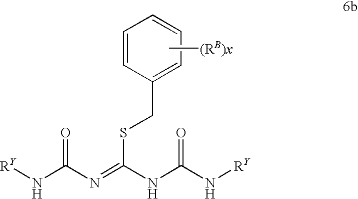

wherein,

x is 0-5;

R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO₂N(R^{J})₂, —COCOR^{J};

R^{J} is hydrogen or unsubstituted C₁₋₆ aliphatic; and

each R^{Y} is C₁₋₆ aliphatic;

comprising:

ic) reacting a compound of formula 6aaa or a suitable salt thereof:

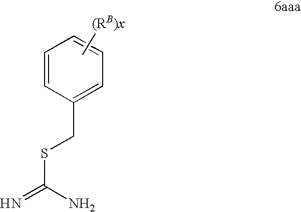

wherein x is 0-5;

R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO₂N(R^{J})₂, —COCOR^{J}; and

R^{J} is hydrogen or unsubstituted C₁₋₆ aliphatic;

with an isocyanate of formula R^{Y}—N═C═O, wherein

R^{Y} is C₁₋₆ aliphatic;

in a suitable mixture of water and an organic solvent to provide a compound of formula 6b.

In another aspect, the present invention also provides compounds of formula 6b useful as intermediates in the processes of the present invention:

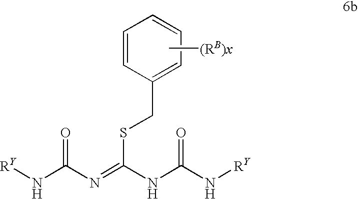

wherein

x is 0-5;

R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO₂N(R^{J})₂, —COCOR^{J};

R^{J} is hydrogen or unsubstituted C₁₋₆ aliphatic; and

each R^{Y} is C₁₋₄ aliphatic.

In yet another aspect, the present invention also provides compounds of formula 4a useful as intermediates in the processes of the present invention:

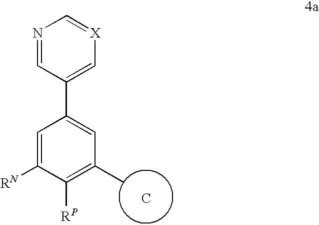

wherein
Ring C is a 6-membered heteroaryl ring having 1-2 nitrogens, wherein:
Ring C is substituted with 1-3 R¹ groups;
   each R¹ is independently selected from OR² or halogen;
   R² is C₁₋₄ aliphatic; or
Ring C is an unsubstituted 2-pyrimidine ring;
   X is nitrogen, CH, or CF; and
   R^{N} and R^{P} are independently NO₂ or NH₂ or NHR^{W};
   R^{W} is an amino protecting group;
      provided that the following compounds are excluded;
      2-nitro-6-(pyridin-2-yl)-4-(pyridin-3-yl)phenyl)amine; 3-(pyridin-2-yl)-5-(pyridin-3-yl)benzene-1,2-diamine; and (2-nitro-3-(pyrazol-1-yl)-5-(pyridin-3-yl)phenyl)amine.

### Definitions and General Terminology

As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention. When more than one position in any given structure may be substituted with more than one substituent selected from a specified group, the substituent may be either the same or different at each position.

If a substituent radical or structure is not identified or defined as “optionally substituted,” the substituent radical or structure is unsubstituted.

Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-6 aliphatic carbon atoms. In some embodiments aliphatic groups contain 1-4 aliphatic carbon atoms. Suitable aliphatic groups include, but are not limited to, linear or branched, substituted or unsubstituted alkyl, alkenyl, or alkynyl groups. Further examples of aliphatic groups include, but are not limited to, methyl, ethyl, propyl, butyl, isopropyl, isobutyl, sec-butyl, tert-butyl, n-pentyl or n-hexyl. The terms “alkyl” and the prefix “alk-”, as used herein, are inclusive of both straight chain and branched saturated carbon chain.

The term “alkoxy”, as used herein, refers to an alkyl group, as previously defined, attached to the principal carbon chain through an oxygen (“alkoxy”) atom.

The terms “haloalkoxy” or “haloalkyl” refers to alkyl, alkenyl or alkoxy, as the case may be, substituted with one or more halogen atoms.

The term “halogen” or “halo”, as used herein, refers to fluorine, chlorine, bromine or iodine.

The term “heteroaryl,” refers to a monocyclic ring system having a total of six ring members containing one or more nitrogen heteroatoms. The term “heteroaryl” may be used interchangeably with the term “heteroaryl ring.”

Without limitation, monocyclic heteroaryl rings include the following: pyridinyl (e.g., pyrid-2-yl, pyrid-3-yl, or pyrid-4-yl); pyrimidinyl (e.g., pyrimidin-2-yl, pyrimidin-4-yl, or pyrimidin-5-yl); pyridazinyl (e.g., pyridazin-3-yl, pyridazin-4-yl, pyridazin-5-yl, or pyridazin-6-yl); pyrazinyl. Heteroaryls are numbered according to standard chemical nomenclature.

In some embodiments, a heteroaryl group may contain one or more substituents. Suitable substituents on the unsaturated carbon atom of a heteroaryl group are selected from those listed in the definition of R¹.

As described herein, a bond drawn from a substituent to the center of one ring within a multiple-ring system (as shown below) represents substitution of the substituent at any substitutable position in any of the rings within the multiple ring system. For example, Figure a represents possible substitution in any of the positions shown in Figure b.

The term “protecting group,” as used herein, represent those groups intended to protect a functional group, such as, for example, an alcohol, amine, carboxyl, carbonyl, etc., against undesirable reactions during synthetic procedures. Commonly used protecting groups are disclosed in Greene and Wuts, Protective Groups in Organic Synthesis, 3^{rd} Edition (John Wiley & Sons, New York, 1999), which is incorporated herein by reference. Examples of nitrogen protecting groups include acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and chiral auxiliaries such as protected or unprotected D, L or D, L-amino acids such as alanine, leucine, phenylalanine and the like; sulfonyl groups such as benzenesulfonyl, p-toluenesulfonyl and the like; carbamate groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl and the like, arylalkyl groups such as benzyl, triphenylmethyl, benzyloxymethyl and the like and silyl groups such as trimethylsilyl and the like. In one embodiment, N-protecting groups are pivaloyl.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools, probes in biological assays, or gyrase/Topo IV inhibitors with improved therapeutic profile.

In one embodiment, the present invention provides a process for preparing a compound of formula 1:

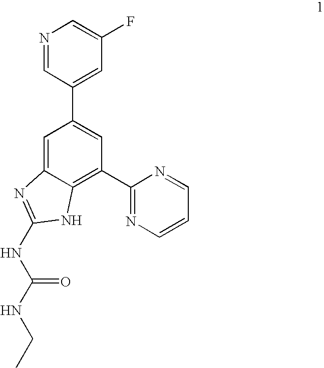

In some embodiments, the process for preparing a compound of formula 1 comprises:

i) providing 3-fluoro-5-(4,4,5,5-tetramethyl-1,3,2-dioxaborolan-2-yl)pyridine (formula 2) and 4-bromo-2-nitro-6-(pyrimidin-2-yl)benzenamine (formula 3),

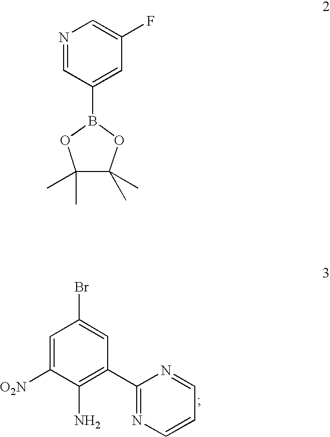

ii) cross coupling the compound of formula 2 and the compound of formula 3 in a biphasic mixture comprising water, an organic solvent, a base and a transition-metal catalyst to produce 4-(5-fluoropyridin-3-yl)-2-nitro-6-(pyrimidin-2-yl)benzenamine of formula 4,

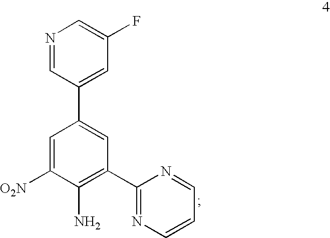

iii) reducing the compound of formula 4 to produce 5-(5-fluoropyridin-3-yl)-3-(pyrimidin-2-yl)benzene-1,2-diamine of formula 5,

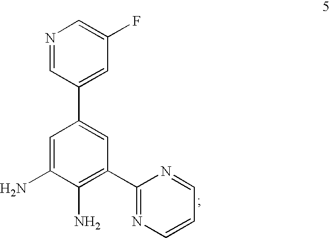

iv) reacting the compound of formula 5 with the compound of formula 6,

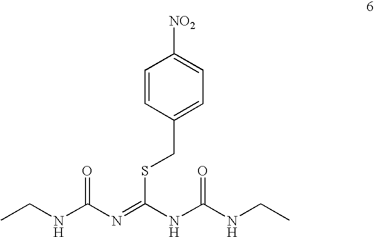

in a biphasic mixture comprising buffered water and an organic solvent to produce the compound of formula 1.

In some embodiments, the organic solvent in ii) is an aprotic solvent.

In some embodiments, the aprotic solvent in ii) is selected from 1,2-dimethoxyethane, dioxane, acetonitrile, toluene, benzene, xylenes, methyl t-butyl ether, methyl ethyl ketone, methyl isobutyl ketone, acetone, N,N-dimethylformamide, N,N-dimethylacetamide, N-methylpyrrolidinone, or dimethylsulfoxide.

In other embodiments, the aprotic solvent in ii) is selected from 1,2-dimethoxyethane or dioxane. In other embodiments, the aprotic solvent in ii) is 1,2-dimethoxyethane.

In other embodiments, the organic solvent in ii) is a protic solvent. In some embodiments, the protic solvent in ii) is selected from methanol, ethanol, or isopropanol.

In some embodiments, the base in ii) is an inorganic base.

In some embodiments, the inorganic base in ii) is selected from potassium carbonate, cesium carbonate, potassium phosphate, sodium carbonate, sodium phosphate, sodium hydroxide, potassium hydroxide or lithium hydroxide.

In some other embodiments, the inorganic base in ii) is selected from potassium carbonate, cesium carbonate or potassium phosphate. In yet other embodiments, the inorganic base in ii) is selected from potassium carbonate.

In some embodiments, the transition-metal catalyst in ii) is a palladium-based catalyst.

In some embodiments, the palladium-based catalyst in ii) is selected from palladium(II)acetate, tetrakis(triphenylphosphine)palladium(0) or tris(dibenzylideneacetone)dipalladium(0). In yet other embodiments, the palladium-based catalyst in ii) is palladium(II)acetate.

In some embodiments, the biphasic mixture in ii) additionally comprises a phosphine ligand.

In some embodiments, the phosphine ligand in ii) is a triarylphosphine ligand or a trialkylphosphine ligand.

In some embodiments, the phosphine ligand in ii) is a trialkylphosphine ligand.

In other embodiments, the trialkylphosphine ligand in ii) is selected from tri-n-butylphosphine, tri-t-butylphosphine or tricyclohexylphosphine.

In some embodiments, the phosphine ligand in ii) is a triarylphosphine ligand selected from triphenylphosphine, tri-o-tolylphosphine, tri-m-tolylphosphine, tri-p-tolylphosphine, or tri-p-anisylphosphine.

In other embodiments, the triarylphosphine ligand in ii) is selected from triphenylphosphine.

In some embodiments, the biphasic mixture in ii) additionally comprises an arsine ligand. In some embodiments, the arsine ligand in ii) is a triarylarsine ligand. In some embodiments, the triarylarsine ligand in ii) is triphenylarsine.

In some other embodiments, the biphasic mixture in ii) additionally comprises a phase transfer catalyst.

In some embodiments, the phase transfer catalyst in ii) is selected from cetyltrimethylammonium bromide, tri-n-butylammonium chloride or benzyltrimethylammonium hydroxide.

In other embodiments, the phase transfer catalyst in ii) is cetyltrimethylammonium bromide.

In some embodiments, the cross coupling reaction of ii) is run at between 75° C. to 100° C.

In other embodiments, the cross coupling reaction of ii) is run at between 80° C. to 90° C. In yet other embodiments, the cross coupling reaction of ii) is run at 85° C.

In some embodiments in iii) the compound of formula 4 is reduced under catalytic hydrogenation conditions comprising a suitable hydrogen atmosphere and a suitable organic solvent.

In other embodiments in iii) the catalytic hydrogenation conditions comprise a palladium metal catalyst on carbon, a hydrogen atmosphere of between 1 to 10 atmospheres and an organic solvent selected from an aprotic solvent, a protic solvent or mixtures thereof.

In other embodiments in iii) the palladium metal catalyst is between 1% to 30% by weight palladium metal on carbon, the hydrogen atmosphere is between 1 to 6 atmospheres and the organic solvent is a protic solvent selected from methanol or ethanol or an aprotic solvent selected from N,N-dimethylacetamide or N,N-dimethylformamide.

In yet other embodiments in iii) the palladium metal catalyst is between 1% to 30% by weight palladium metal on carbon, the hydrogen atmosphere is between 1 to 6 atmospheres and the organic solvent is a protic solvent selected from methanol or ethanol or an aprotic solvent selected from N,N-dimethylacetamide.

In other embodiments in iii) the palladium metal catalyst is between 5% to 10% by weight palladium metal on carbon, the hydrogen atmosphere is between 1 to 5 atmospheres and the organic solvent is N,N-dimethylacetamide.

In some embodiments, the organic solvent in iv) is an aprotic solvent.

In some embodiments, the aprotic solvent in iv) is selected from 1,2-dimethoxyethane or dioxane.

In some embodiments, the compound of formula 6 is used as a solution in an aprotic solvent.

In some embodiments, the compound of formula 6 is used as a solution in 1,2-dimethoxyethane.

In some other embodiments, the compound of formula 6 is used in a solid form.

In some embodiments, the biphasic buffered water solution in iv) comprises an aprotic solvent and an aqueous buffer adjusted to a pH of between 2 to 5.

In some embodiments, the biphasic buffered water solution in iv) comprises an aprotic solvent and an aqueous buffer adjusted to a pH of between 3 to 4.

In some embodiments, the aprotic solvent in iv) is selected from 1,2-dimethoxyethane or dioxane.

In other embodiments, the aprotic solvent in iv) is 1,2-dimethoxyethane.

In some embodiments, the reaction of iv) is run at between 50° C. to 100° C.

In other embodiments, the reaction of iv) is run at between 70° C. to 90° C.

In yet other embodiments, the reaction of iv) is run at between 75° C. to 85° C. In some embodiments, the process for preparing 1-ethyl-3-(5-(5-fluoropyridin-3-yl)-7-(pyrimidin-2-yl)-1H-benzo[d]imidazol-2-yl)urea of formula 1 further comprises:

a) slurrying a preparation comprising a compound of formula 1 in an organic solvent, water and acid to obtain a suspension of the compound of formula 1,

b) heating the suspension to obtain a homogeneous solution of compound 1,

c) filtering the homogeneous solution of compound 1,

d) cooling the solution to obtain a salt of compound 1 in a solid form.

In some embodiments, said organic solvent in a) is a protic solvent, said acid is a sulfonic acid and said heating in b) is to a temperature of between 40° C. to 90° C.

In some other embodiments, said protic solvent in a) is methanol or ethanol, said sulfonic acid is methanesulfonic acid or ethanesulfonic acid and said heating in b) is to a temperature of between 60° C. to 85° C.

In some embodiments, the process for preparing 1-ethyl-3-(5-(5-fluoropyridin-3-yl)-7-(pyrimidin-2-yl)-1H-benzo[d]imidazol-2-yl)urea of formula 1 further comprises:

e) recrystallizing the salt of compound 1 in a suitable organic solvent or a mixture of an organic solvent and water at a suitable temperature.

In some embodiments, the recrystallization in e) comprises a mixture of water and a protic solvent.

In other embodiments in e), the protic solvent is ethanol.

In some embodiments, the present invention provides a process for purifying a compound of formula 1,

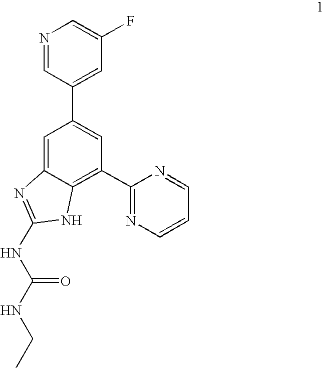

comprising:

a) slurrying the compound of formula 1 in an organic solvent, water and acid to obtain a suspension of the compound of formula 1,

b) heating the suspension to obtain a homogeneous solution of compound 1,

c) filtering the homogeneous solution of compound 1,

d) cooling the solution to obtain a salt of compound 1 in a solid form.

In some embodiments of the process to purify the compound of formula 1, in a) said organic solvent is a protic solvent and said acid is a sulfonic acid and in b) said heating is to a temperature of between 40° C. to 90° C.

In some other embodiments of the process to purify the compound of formula 1, in a) said protic solvent is methanol or ethanol and said sulfonic acid is methanesulfonic acid or ethanesulfonic acid and in b) said heating is to a temperature of between 50° C. to 80° C.

In some other embodiments of the process to purify the compound of formula 1, in a) said protic solvent is ethanol and said sulfonic acid is ethanesulfonic acid and in b) said heating is to a temperature of between 60° C. to 70° C.

In some embodiments, the present invention provides a process of purifying the compound of formula 1, further comprising recrystallizing the salt of compound 1 in a suitable organic solvent or a mixture of an organic solvent and water at a suitable temperature.

In some embodiments, the recrystallization comprises a mixture of water and a protic solvent. In other embodiments, the protic solvent is ethanol.

In other embodiments, the process for preparing a compound of formula 1 comprises:

ia) reacting 5-(5-fluoropyridin-3-yl)-3-(pyrimidin-2-yl)benzene-1,2-diamine of formula 5,

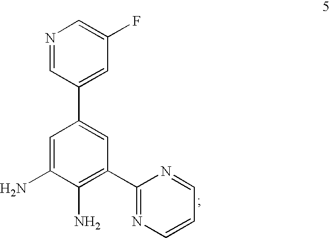

with the compound of formula 6,

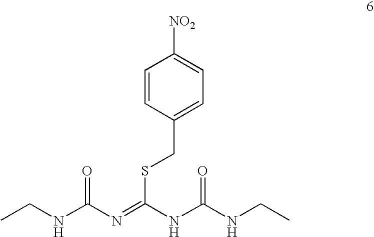

in a biphasic mixture comprising buffered water and an organic solvent to produce the compound of formula 1.

In some embodiments, the organic solvent in ia) is an aprotic solvent.

In some embodiments, the aprotic solvent in ia) is selected from 1,2-dimethoxyethane or dioxane.

In some embodiments, the compound of formula 6 is obtained as a solution in an aprotic solvent.

In some embodiments, the compound of formula 6 is obtained as a solution in 1,2-dimethoxyethane.

In some other embodiments, the compound of formula 6 is obtained in a solid form.

In some embodiments, the biphasic buffered water solution in ia) comprises an aprotic solvent and an aqueous buffer adjusted to a pH of between 2 to 5.

In some embodiments, the biphasic buffered water solution in ia) comprises an aprotic solvent and an aqueous buffer adjusted to a pH of between 3 to 4.

In some embodiments, the aprotic solvent in ia) is selected from 1,2-dimethoxyethane or dioxane.

In other embodiments, the aprotic solvent in ia) is 1,2-dimethoxyethane.

In some embodiments, the reaction of ia) is run at between 50° C. to 100° C. In other embodiments, the reaction of ia) is run at between 70° C. to 90° C. In yet other embodiments, the reaction of ia) is run at between 75° C. to 85° C. In other embodiments, the present invention provides a process for preparing compounds of formula I:

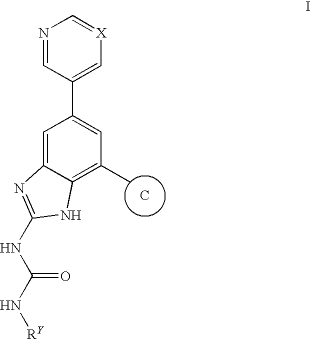

comprising:

ib) reacting a compound of formula 4b,

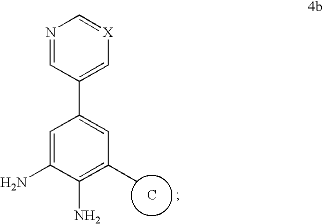

wherein,
Ring C is a 6-membered heteroaryl ring having 1-2 nitrogens, wherein:
Ring C is substituted with 1-3 R¹ groups;
   each R¹ is independently selected from OR² or halogen; and
   R² is C₁₋₄ aliphatic; or
Ring C is an unsubstituted 2-pyrimidine ring;
   X is nitrogen, CH, or CF;:
   with a compound of formula 6b,

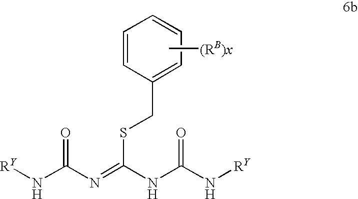

wherein,
x is 0-5;
R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO₂N(R^{J})₂, —COCOR^{J}; wherein
R^{J} is hydrogen or unsubstituted C₁₋₆ aliphatic; and
each R^{Y} is C₁₋₆ aliphatic.
   in a biphasic mixture comprising buffered water and an organic solvent to produce the compound of formula I.

In some embodiments, the organic solvent in ib) is an aprotic solvent.

In some embodiments, the aprotic solvent in ib) is selected from 1,2-dimethoxyethane or dioxane.

In some embodiments, the compound of formula 6b is obtained as a solution in an aprotic solvent.

In some embodiments, the compound of formula 6b is obtained as a solution in 1,2-dimethoxyethane.

In some other embodiments, the compound of formula 6b is obtained in a solid form.

In some embodiments, the biphasic buffered water solution in ib) comprises an aprotic solvent and an aqueous buffer adjusted to a pH of between 2 to 5.

In some embodiments, the biphasic buffered water solution in ib) comprises an aprotic solvent and an aqueous buffer adjusted to a pH of between 3 to 4.

In some embodiments, the aprotic solvent in ib) is selected from 1,2-dimethoxyethane or dioxane.

In other embodiments, the aprotic solvent in ib) is 1,2-dimethoxyethane.

In some embodiments, the reaction of ib) is run at between 50° C. to 100° C.

In other embodiments, the reaction of ib) is run at between 70° C. to 90° C.

In yet other embodiments, the reaction of ib) is run at between 75° C. to 85° C.

In other embodiments, the present invention provides a process for preparing compounds of formula 6b,

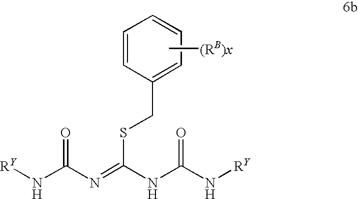

wherein,

x is 0-5;

R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO2N(R^{J})₂, —COCOR^{J};

R^{J} is hydrogen or unsubstituted C₁₋₆ aliphatic; and

each R^{Y} is C₁₋₆ aliphatic;

comprising:
ic) reacting a compound of formula 6aaa or a suitable salt thereof:

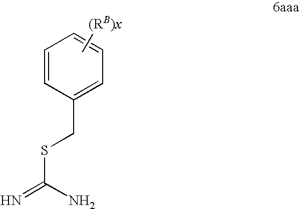

wherein x is 0-5;

R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO₂N(R^{J})₂, —COCOR^{J}; and

R^{J} is hydrogen or unsubstituted C₁₋₆ aliphatic;

with an isocyanate of formula R^{Y}NCO, wherein

R^{Y} is C₁₋₆ aliphatic;

in a suitable mixture of water and an organic solvent to provide a compound of formula 6b.

In some embodiments of ic), the organic solvent is an aprotic solvent. In other embodiments, the aprotic solvent is selected from 1,2-dimethoxyethane, dioxane, acetonitrile, toluene, benzene, xylenes, methyl t-butyl ether, methyl ethyl ketone, methyl isobutyl ketone, acetone, N,N-dimethylformamide, N,N-dimethylacetamide, N-methylpyrrolidinone, or dimethylsulfoxide.

In other embodiments, the aprotic solvent in ic) is selected from 1,2-dimethoxyethane or dioxane. In other embodiments, the aprotic solvent in ic) is 1,2-dimethoxyethane.

In some embodiments, the compounds of formula 5 or formula 4b and the compounds of formula 6 or formula 6b are prepared according to the processes and procedures disclosed herein. In other embodiments, the compounds of formula 4b are prepared according to procedures known to one of skill in the art (e.g., see WO 02/060879, WO 05/0122292, US2005/0038247, US2006/0122196 and WO 07/056330 each of which is incorporated by reference for the procedures related to the preparation of compounds of formula 4b).

In some embodiments, the present invention provides compounds of formula 6b:

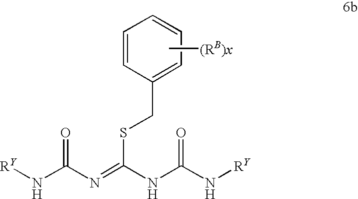

wherein

x is 0-5;

R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO2N(R^{J})₂, —COCOR^{J};

R^{J} is hydrogen or unsubstituted C₁₋₆ aliphatic; and

each R^{Y} is C₁₋₆ aliphatic.

In some embodiments of compounds of formula 6b, x is 1, the R^{B} substituent is para and R^{Y} is C₁₋₄ aliphatic.

In other embodiments of compounds of formula 6b, the R^{B} substituent is para NO₂ and R^{Y} is ethyl.

In yet other embodiments, the present invention provides compounds of formula 4a:

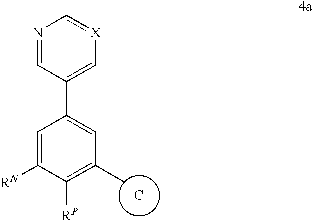

wherein
Ring C is a 6-membered heteroaryl ring having 1-2 nitrogens, wherein:
Ring C is substituted with 1-3 R¹ groups;
   each R¹ is independently selected from OR² or halogen; and
   R² is C₁₋₄ aliphatic; or
Ring C is an unsubstituted 2-pyrimidine ring;
   X is nitrogen, CH, or CF; and
   R^{N} and R^{P} are independently NO₂, NH₂ or NHR^{W};
   R^{W}is an amino protecting group;
      provided that the following compounds are excluded;
      2-nitro-6-(pyridin-2-yl)-4-(pyridin-3-yl)phenyl)amine; 3-(pyridin-2-yl)-5-(pyridin-3-yl)benzene-1,2-diamine; and (2-nitro-3-(pyrazol-1-yl)-5-(pyridin-3-yl)phenyl)amine.

In some embodiments of compounds of formula 4a, X is C—F; Ring C is unsubstituted pyrimidine, R^{N} and R^{P} are both NH₂.

In other embodiments of compounds of formula 4a, X is C—F; Ring C is unsubstituted pyrimidine, R^{N} is NO₂ and R^{P} is NH₂.

In yet other embodiments of compounds of formula 4a, X is C—F; Ring C is unsubstituted pyrimidine, R^{N} is NH₂ and R^{P} is NO₂.

In other embodiments, the present invention provides compounds of formula 4a:

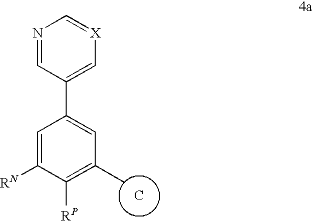

wherein
Ring C is a 6-membered heteroaryl ring having 1-2 nitrogens, wherein:
Ring C is substituted with 1-3 R¹ groups;
   each R¹ is independently selected from OR² or halogen; and
   R² is C₁₋₄ aliphatic; or
Ring C is an unsubstituted 2-pyrimidine ring;
   X is nitrogen, CH, or CF; and
   R^{N} and R^{P} are independently NO₂, NH₂ or NHR^{W};
   R^{W} is an amino protecting group.

In other embodiments, the present invention provides compounds of formula 4a:

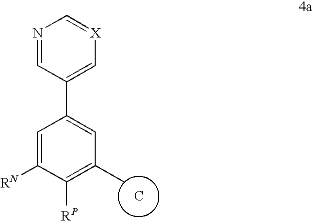

wherein
Ring C is a 6-membered heteroaryl ring having 1-2 nitrogens, wherein:
Ring C is substituted with 1-3 R¹ groups;
   each R¹ is independently selected from OR² or halogen; and
   R² is C₁₋₄ aliphatic; or
Ring C is an unsubstituted 2-pyrimidine ring;
   X is nitrogen, CH, or CF; and
   R^{N} and R^{P} are independently NO₂ or NH₂;
   provided that the following compounds are excluded;
   2-nitro-6-(pyridin-2-yl)-4-(pyridin-3-yl)phenyl)amine; 3-(pyridin-2-yl)-5-(pyridin-3-yl)benzene-1,2-diamine; and (2-nitro-3-(pyrazol-1-yl)-5-(pyridin-3-yl)phenyl)amine.

In other embodiments, the present invention provides compounds of formula 4a:

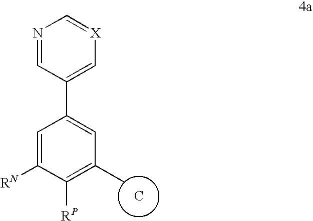

wherein
Ring C is a 6-membered heteroaryl ring having 1-2 nitrogens, wherein:
Ring C is substituted with 1-3 R¹ groups;
   each R¹ is independently selected from OR² or halogen; and
   R² is C₁₋₄ aliphatic; or
Ring C is an unsubstituted 2-pyrimidine ring;
   X is nitrogen, CH, or CF; and
   R^{N} and R^{P} are independently NO₂ or NH₂.

### Processes and Intermediates

The following definitions describe terms and abbreviations used herein:
Ac acetyl
Bu butyl
Et ethyl
Ph phenyl
Me methyl
THF tetrahydrofuran
DCM dichloromethane
CH₂Cl₂ dichloromethane
EtOAc ethyl acetate
CH₃CN acetonitrile
EtOH ethanol
MeOH methanol
MTBE methyl tert-butyl ether
DMF N,N-dimethylformamide
DMA N,N-dimethylacetamide
DMSO dimethyl sulfoxide
HOAc acetic acid
TFA trifluoroacetic acid
Et₃N triethylamine
DIPEA diisopropylethylamine
DIEA diisopropylethylamine
K₂CO₃ potassium carbonate
Na₂CO₃ sodium carbonate
Cs₂CO₃ cesium carbonate
NaHCO₃ sodium bicarbonate
NaOH sodium hydroxide
Na₂SO₄ sodium sulfate
K₃PO₄ potassium phosphate
NH₄Cl ammonium chloride
LC/MS liquid chromatography/mass spectra
HPLC high performance liquid chromtagraphy
GC gas chromatography
LC liquid chromatography
Hr or h hours
atm atmospheres
rt or RT room temperature
TLC thin layer chromatography
HCl hydrochloric acid
H₂O water
EtNCO ethyl isocyanate
Pd/C palladium on carbon
NaOAc sodium acetate
H₂SO₄ sulfuric acid
N₂ nitrogen gas
H₂ hydrogen gas
n-BuLi n-butyl lithium
Piv pivaloyl
DI de-ionized
Pd(OAc)₂ palladium(II)acetate
PPh₃ triphenylphosphine
i-PrOH isopropyl alcohol
NBS N-bromosuccinimide
Pd[(Ph₃)P]₄ tetrakis(triphenylphosphine)palladium(0)
PTFE polytetrafluoroethylene
NLT not less than
NMT not more than
rpm revolutions per minute
SM starting material
Equiv. equivalents
¹H NMR proton nuclear magnetic resonance

As used herein, other abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997, herein incorporated in its entirety by reference.

In one embodiment, the invention provides a process and intermediates for preparing a compound of formula 6 as outlined in Scheme I.

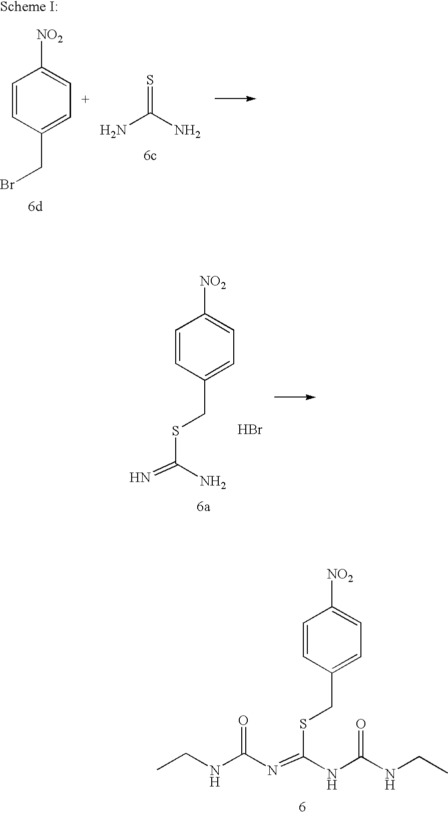

In Scheme I, isothiourea of formula 6a is prepared by addition of thiourea of formula 6c in a suitable solvent (e.g., acetone) to a mixture of the bromide of formula 6d in a suitable solvent (e.g., acetone). Treatment of isothiourea of formula 6a with excess ethylisocyanate in a suitable mixture of water and an organic solvent (e.g., 1,2-dimethoxyethane) afforded the pseudothiourea of formula 6.

In one embodiment of Scheme I, pseudothiourea of formula 6 is prepared by reaction of commercially available 2-(4-nitrobenzyl)isothiourea hydrobromide salt of formula 6a with excess ethyl isocyanate in a mixture of water and an organic solvent (e.g., 1,2-dimethoxyethane) at a temperature of between 0° C. to 50° C.

In one embodiment, the compound of formula 6 is prepared as a solution in an organic solvent (e.g., 1,2-dimethoxyethane) and used without further isolation in the preparation of compound 1 as described in Scheme V.

In another embodiment, the compound of formula 6 is isolated as a solid and used in the preparation of compound 1 as described in Scheme V.

In another embodiment, the invention provides a process and intermediates to prepare a compound of formula 6b as described below in Scheme Ia.

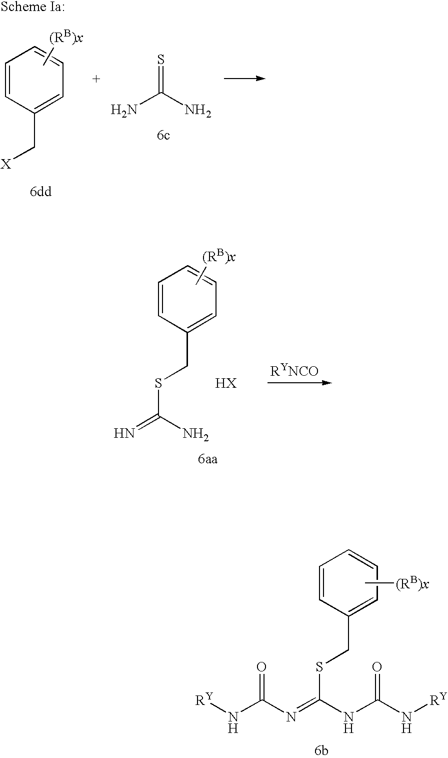

In Scheme Ia, isothiourea of formula 6b is prepared by addition of thiourea of formula 6c in a suitable solvent (e.g., acetone) to a mixture of the compound of formula 6dd in a suitable solvent (e.g., acetone). Treatment of isothiourea intermediate of formula 6aa with excess isocyanate in a suitable mixture of water and an organic solvent (e.g., 1,2-dimethoxyethane) affords a pseudothiourea of formula 6b. In Scheme Ia above, radicals R^{B}, x, and R^{Y} are as defined herein. It will be appreciated that radical HX in compounds of formula 6aa represents a suitable salt (e.g., such as the hydrobromide salt) which is optionally present to aid in the isolation of compound 6aa. Radical X in compounds of formula 6dd is a suitable leaving group (e.g., halogen). As used herein, a suitable leaving group is a chemical moiety that is readily displaced by a desired incoming chemical moiety. Suitable leaving groups are well known in the art, e.g., see, “Advanced Organic Chemistry,” Jerry March, 4^{th} Ed., pp. 351-357, John Wiley and Sons, N.Y. (1992) and “Comprehensive Organic Transformations,” Larock, Richard C., 2^{nd} Ed., John Wiley & Sons, 1999, the contents both of which are incorporated herein by reference.

Such leaving groups include, but are not limited to, halogen, sulphonyloxy, optionally substituted alkylsulphonyl, optionally substituted alkenylsulfonyl, optionally substituted arylsulfonyl, and diazonium moieties.

In another embodiment, the invention provides a process and intermediates for preparing a compound of formula 2 as outlined in Scheme II.

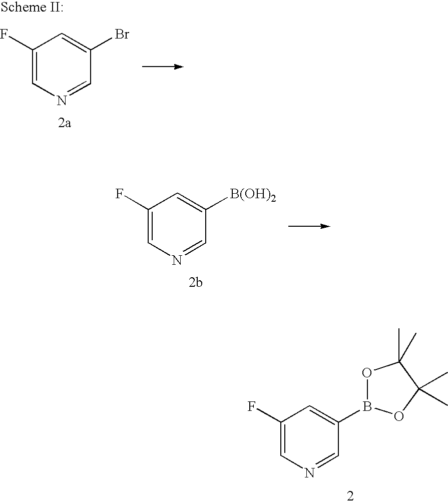

In Scheme II, the boronic acid of formula 2b is prepared by reaction of commercially available 3-bromo-5-fluoropyridine of formula 2a with a strong lithium base (e.g., n-butyl lithium) in the presence of a borate ester (e.g., isopropyl borate) in a suitable aprotic solvent. Suitable aprotic solvents include, for example, tetrahydrofuran. Subsequently, the intermediate borate ester mixture is quenched and hydrolyzed with an aqueous mineral acid (e.g., 9% aqueous HCl) to give boronic acid 2b. Subsequently, the boronic acid of formula 2b is esterified with pinacolate alcohol in a suitable solvent (e.g., toluene) at an elevated temperature of between 80° C. to 150° C. to give the compound of formula 2.

In one embodiment, a compound of formula 2 in Scheme II can be purchased commercially.

In one embodiment, a compound of formula 3 in Scheme III can be prepared from a compound of formula 3c according to the procedures described in WO 05/012292. In another embodiment, the compound of formula 3c may be purchased commercially. In yet another embodiment, the compound of formula 3c can be prepared according to known procedures from 2-bromobenzeneamine.

In another embodiment, the invention provides a process and intermediates for preparing a compound of formula 3 as outlined in Scheme III.

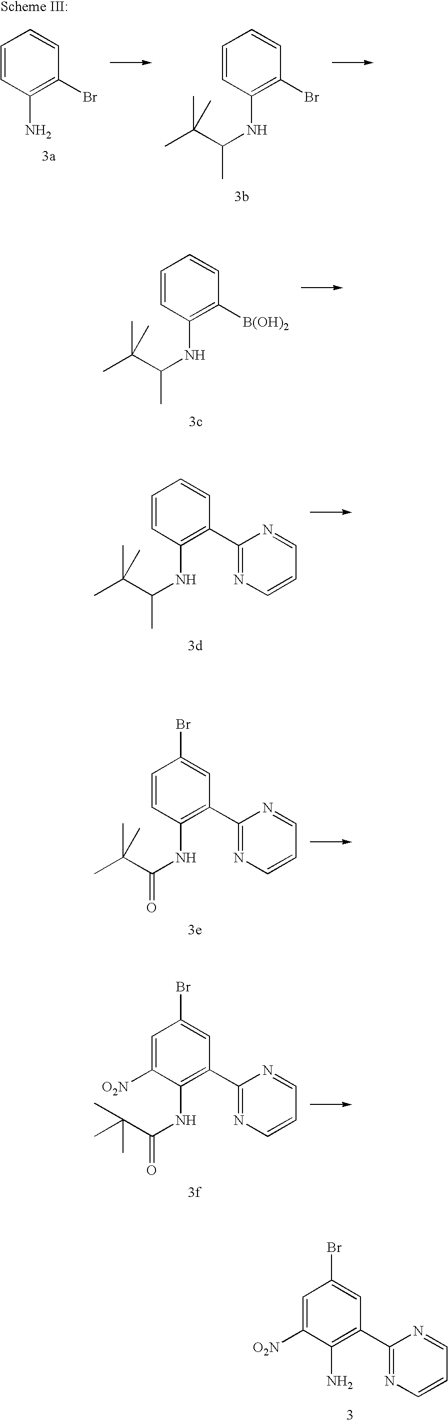

Referring to scheme III, the bromoaniline of pivalamide of formula 3b is prepared by treating a compound of formula 3a with pivaloylchloride in a suitable aprotic solvent (e.g., dichloromethane) in the presence of a suitable base (e.g., an organic tertiary amine base such as triethylamine) at temperatures between −20° C. and 25° C. Preparation of the corresponding boronic acid of formula 3c is achieved by reacting a bromide of formula 3b with a strong lithium base (e.g., n-butyl lithium) in a suitable aprotic solvent (e.g., tetrahydrofuran) followed by addition of a suitable borate ester (e.g., isopropylborate) at a suitable temperature (e.g., −45° C. to 100° C.). Biaryl intermediate 3d is prepared by cross coupling reaction of boronic acid 3c with 2-chloropyrimidine in a biphasic mixture of aqueous inorganic base (e.g., an alkali metal base such as sodium carbonate) and a suitable organic solvent (e.g., glycol dimethyl ether) with a suitable transition-metal catalyst (e.g., tetrakis(triphenylphosphine)palladium(0)) at a suitable temperature (e.g., between 25° C. to 120° C.). Compound of formula 3d is then brominated with a brominating reagent (e.g., NBS or N-bromosuccinimide) in a glacial acetic acid at a suitable temperature (e.g., 25° C. to 80° C.) to provide aryl bromide of formula 3e. Nitration of 3e is accomplished by reacting a cooled aqueous solution (e.g., between −20° C. to 10° C.) of 3e in a suitable acid (e.g., sulfuric acid) with nitric acid to provide a compound of formula 3f. Removal of the pivaloyl protecting group is achieved with a suitable acid (e.g., hydrochloric acid) in an organic solvent (e.g., absolute ethanol) at a suitable temperature (e.g., between 30° C. to 120° C.) to yield a compound of formula 3.

In another embodiment, the invention provides a process and intermediates for preparing a compound of formula 4 as outlined in Scheme IV.

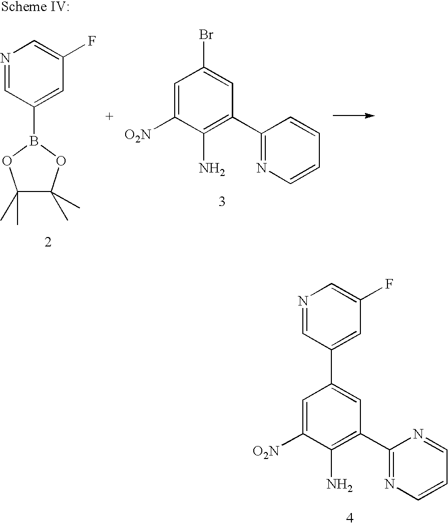

In Scheme IV, boronate of formula 2 is cross coupled with aryl bromide of formula 3 in a biphasic mixture of aqueous inorganic base (e.g., an alkali metal base such as potassium carbonate, cesium carbonate) and a suitable aprotic organic solvent (e.g., 1,2-dimethoxyethane or dioxane) with a suitable transition-metal catalyst (e.g., palladium(II)acetate), a suitable phosphine catalyst (e.g., triphenylphosphine) and a suitable phase transfer catalyst (e.g., cetyltrimethylammonium bromide) at a suitable temperature (e.g., between 40° C. to 120° C.) to yield a compound of formula 4.

In another embodiment, in Scheme IV, boronate of formula 2 is cross coupled with aryl bromide of formula 3 in a biphasic mixture of inorganic base (e.g., an alkali metal base such potassium phosphate) and a suitable protic organic solvent (e.g., ethanol) with a suitable transition-metal catalyst (e.g., palladium(II)acetate), a suitable phosphine catalyst (e.g., triphenylphosphine) and a suitable phase transfer catalyst (e.g., cetyltrimethylammonium bromide) at a suitable temperature (e.g., between 40° C. to 120° C.) to yield a compound of formula 4.

In another embodiment, the invention provides a process and intermediates for preparing a compound of formula 5 as outlined in Scheme V.

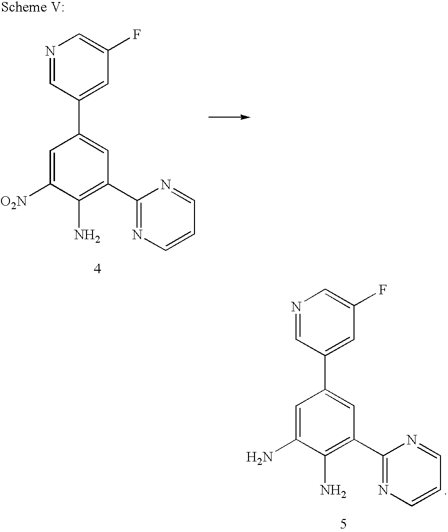

In Scheme V, conversion of the nitro group in the compound of formula 4 to the aniline may be achieved under reducing conditions (e.g., a suitable hydrogen atmosphere in the presence of a palladium catalyst) in a suitable solvent (e.g., N,N-dimethylacetamide) to give the desired diamine of formula 5.

In another embodiment, the invention provides a process and intermediates for preparing a compound of formula 1 as outlined in Scheme VI.

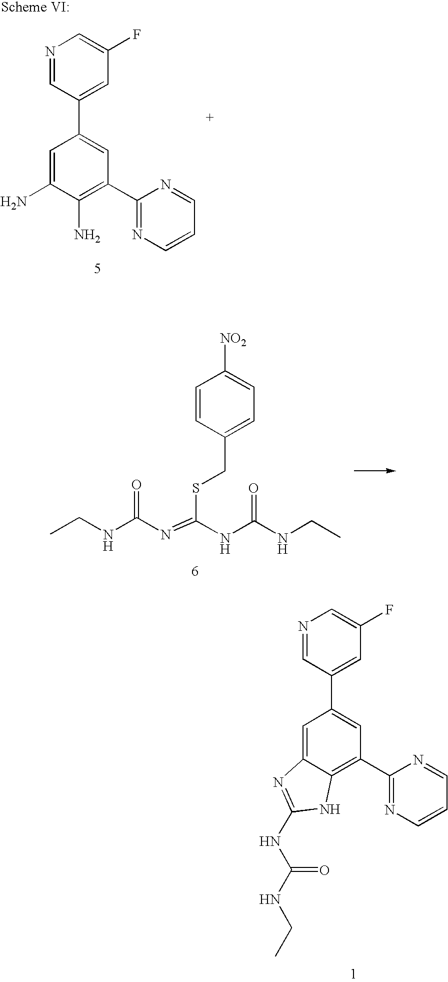

In Scheme VI, the diamine of formula 5 in an acidic aqueous solution (e.g., sodium acetate in water with the pH adjusted to between 3 to 4 with a suitable acid such as, for example, concentrated sulfuric acid) is reacted with a solution of compound of formula 6 in an organic solvent (e.g., 1,2-dimethoxyethane) at a suitable temperature (e.g., between 40° C.-120° C.).

### EXAMPLES

The following preparative examples are set forth in order that this invention is more fully understood. These examples are for the purpose of illustration only and are not to be construed as limiting the scope of the invention in any way.

### Analytical Methods Used

(A) HPLC on a Waters XBridge Phenyl column, 4.6×75 mm, 3.5 microns. Mobile phase A is water/1 M ammonium formate, pH 7.0 (99: 1). Mobile phase B is ACN/Water/1 M ammonium formate, pH 7.0 (90:9:1). Gradient 10 to 100% B in 10 to 12 min. Flow rate 1.2 mL/min. Detection at UV 245 nm. T=30° C.

(B) LC on an Agilent RP18, 4.6×250 mm column. Mobile phase ACN/H₂O/TFA (60:40:0.1). Detection at 265 nm. Flow rate 1.0 mL/min. Run time 22 min.

(C) GC on an HP-5 column. Using H₂ as the carrier gas and a temperature gradient of 8-2-10-240. Flow rate 1.4 mL/min. Run time 24 min.

(D) HPLC on an Altima C18 4.6×250 mm column. Mobile phase ACN/H₂O (7:3). Detection at 220 nm. Flow rate 1.0 mL/min. Ambient temp. Run time 21 min.

(E) Same as (D) with mobile phase ACN/H₂O/TFA (70:30:0.1) and detection at 250 nm.

(F) LC on an Agilent HC-C18 4.6×250 mm column. Mobile phase ACN/H₂O/TFA (50:50:0.1). Detection 250 nm. Flow rate 1.0 mL/min. Ambient temp. Run time 25 min.

(G) same as (E) but with a lower flow rate of 0.8 ml/min and mobile phase 70:30:0.1%.

(H) GC on a J&W DB-1 column, 60 m×0.32 mm i.d., 3.0 mm film thickness. Carrier gas He. Run time 17.0 min. Initial temperature 40° C., hold at 40° C. for 5 min. Ramp to 100° C. (10° C./min), then ramp to 240° C. (35° C./min) and hold at 240° C. for 2 min. Column flow 2.5 mL/min Helium (constant). FID detector. Split ratio 30:1.

### Example 1

### 4-(5-fluoropyridin-3-yl)-2-nitro-6-(pyrimidin-2-yl)benzenamine (4)

### Method 1

A 3 L round-bottom flask was fitted with a mechanical overhead stirrer, a reflux condenser, and a thermometer and purged with N₂. 3-Fluoro-5-(4,4,5,5-tetramethyl-1,3,2-dioxaborolan-2-yl)pyridine (2) (95.0 g, 421 mmol, 1.2 equiv), 4-bromo-2-nitro-6-(pyrimidin-2-yl)benzenamine (3) (104.55 g, 354 mmol, 1.0 equiv), K₃PO₄ (93.1 g, 438 mmol, 1.25 equiv, Riedel-deHaen #S29375-366), Pd(OAc)₂ (0.80 g, 3.5 mmol, 1 mol %, Aldrich #06410JE) cetyltrimethylammonium bromide (1.28 g, 3.5 mmol, 1 mol %, Aldrich #0823CE) and PPh₃ (3.74 g, 14 mmol, 4 mol %, Fluka #1093859 ) were added followed by EtOH (10 vols) and water (1. 1 vols). The heterogeneous reaction mixture was stirred vigorously (>300 rpm) and heated to reflux (80° C.). After 6 hours a sample was removed for analysis of reaction completion (method C). After the reaction was judged complete, the mixture was cooled to 30° C., then water (10 vol) was added and the reaction stirred for 2 h. The resulting slurry was filtered and the filter cake washed with water (2×5 vol) and acetonitrile (2×5 vol). The filter cake was then charged back to the flask and water added (10 vol). The slurry was stirred for 3 h at 30° C. and filtered again. The filter cake was then transferred to a dish and allowed to dry under vacuum at 60-80° C. for 12 h furnishing compound 4 as an orange powder (104.87 g, 88%), purity: 96.14% AUC determined using method A. Typical reaction times were 7.9 min for SM (2) and 6.9 min for product (4).

### Method 2

4-Bromo-2-nitro-6-(pyrimidin-2-yl)benzenamine (3) (754 g, 2.56 moles, 1.0 equiv.), 3-fluoro-5-(4,4,5,5-tetramethyl-1,3,2-dioxaborolan-2-yl)pyridine (2) (684.0 g, 3.04 mmol, 1.2 equiv), K₂CO₃ (3M, 1.06 Kg, 1.59 moles, 3.0 equiv., ca. 3.4 vols), cetyltrimethylammonium bromide (9.70 g, 0.025 moles, 1 mol %), Pd(OAc)2 (5.80 g, 0.025 moles, 1% mol) and PhP₃ (27.0 g, 0.1 moles, 4% mol) were charged to a flask under N₂ equipped with a mechanical stirring apparatus, reflux condenser and a thermometer followed by the addition of DME (7.54 L, 10 vols). The heterogeneous reaction mixture was stirred vigorously (>300 rpm) and heated to reflux (80° C.). During the course of the reaction the mixture became viscous as the amount of product accumulates and after 5 h a sample was removed for analysis (method A). After the reaction was judged completed, water (2.53 L, 5 vols) was added and heating stopped. The reaction mixture was cooled to 25° C. at which point the biphasic mixture was stirred for an additional 2 h. The resulting slurry was filtered and the filter cake washed with 10% vol/vol of ACN in water (3.77 L, 2×3 vols). The filter cake was then transferred to a dish and allowed to dry under vacuum at 60˜80° C. for 12 h. The title compound was obtained as a brown powder (726.2 g, 92%, 96.9% AUC by method A, typical yields ranged between 85-95%).

### Example 2

### 5-(5-fluoropyridin-3-yl)-3-(pyrimidin-2-yl)benzene-1,2-diamine (5)

To a 20 L Büchi hydrogenator equipped with a Büchi gas controller and a Huber temperature controller was added 4-(5-fluoropyridin-3-yl)-2-nitro-6-(pyrimidin-2-yl)benzenamine (4) (1095 g, 3.581 mol, 1.00 equiv.), 5% Pd/C (438 g, 50% wet) and DMA (N,N-dimethylacetamide, 11.0 L, 10 vol). The reactor was purged with nitrogen gas (3 times), then hydrogen gas (3 times). The reaction temperature was set to 23° C. and the pressure of the reactor was set to 50 psi with hydrogen gas. The mixture was agitated at 1350 rpm. The reaction progress was monitored by the hydrogen uptake curve that started to flatten out after 1.7 hours. The hydrogenation was continued for an additional 2 hours. The reactor with purged with nitrogen gas (3 times). Removed an aliquot and analyzed it by HPLC (method A) to obtain NMT 1% AUC of 4. The hydrogenation was continued for another 0.5 h, followed by pressure release and N₂ purge (3 times). The reaction mixture was filtered through a pad of celite (400 g, wetted with 1.50 L of DMA) and a #3 Whatman filter paper. The hydrogenator was washed with DMA (1.10 L) and filtered through the celite pad. The filter cake was washed with 0.300 L of DMA. The resulting filtrates were combined and treated with activated carbon twice (493 g each). After the second wash, the charcoal was removed by filtration, and the filtrate filtered through #3 filter papers. Water (20.0 L) was slowly added to maintain a temperature of NMT 40° C. and a yellow solid precipitated out. Filtered the slurry through a #3 Whatman filter paper at room temperature and washed the yellow solid with 6.0 L of water. The yellow solid was dried in a vacuum oven at 60° C. under vacuum to afford the title compound (5) as a white powder (671 g, 68%, 99.2% AUC using method A). Typical retention times are 7.08 min for SM (4) and 5.03 min for product (5).

### Example 3

### N,N-diethylureamido-2-(4-nitrobenzyl)-2-thiopseudourea (6)

2-(4-nitrobenzyl) isothiourea hydrobromide (6a) (748 g, 2.56 moles, 1.0 equiv.), water (1.0 L, 1.4 vol) and DME (2 L, 2.8 L) were charged into a 10 L Morton flask that was equipped with an overhead stirrer and a thermocouple. Ethyl isocyanate (800 mL, 10.2 moles, 4.0 equiv.) was added to the cleared yellow solution and the resulting biphasic mixture (EtNCO layer and a water/DME layer) was stirred vigorously at 15˜25° C. for 21-24 hours. At the end of the reaction the mixture is comprised of a clear and colorless top aqueous layer and a clear and yellow bottom organic layer. The aqueous layer was removed and DME (2 L, 2.8 vol) was added into the Morton flask, then concentrated to 2.8 vol at 35˜40° C. under vacuum pressure. This distillation was repeated one additional time then the remaining mixture containing compound of formula 6 was used directly in the next step. Before running the next step, the mixture was analyzed (method H) to make sure all the isocyanate had been removed (if this is not the case, additional distillations might be needed).

### Example 3A

### Alternative isolation of N,N-diethylureamido-2-(4-nitrobenzyl)-2-thiopseudourea (6)

To a suspension of 2-(4-nitrobenzyl)isothiourea hydrobromide (6a) (2.15 g, 7.33 mmol) in pH 7.0 buffer solution (3.0 mL) and 1,2-dimethoxyethane (4.0 mL) was added ethyl isocyanate (2.40 mL, 30.6 mmol). The biphasic solution was allowed to stir for 18 hours, at which point a white suspension was observed. The solids were filtered. The filter cake was washed with water then diethyl ether to afford the title compound 6 after air drying as a white solid (1.76 g, 68%). The title compound exists as a mixture of rotamers. ¹H NMR: δ 12.00 (bs, 1NH), 8.15 (d, 2H, J=8.1 Hz), 7.95 bs, 1NH), 7.69 (d, 2H, J=8.3 Hz), 7.64 (app t, 1NH), 4.29 (s, 2H), 3.10 (quintet, 2H, J=6.6 Hz), 3.01 (quintet, 2H, J=7.0 Hz), 1.07 (t, 3H, J=7.1 Hz), 1.00 (t, 3H, J=7.2 Hz) ppm.

### Example 3B

### 2-(4-nitrobenzyl)isothiourea hydrobromide (6a)

To a 72-L reactor under a nitrogen atmosphere was added thiourea (1.55 kg) and acetone (30 L). The mixture was agitated. To a separate flask was added 4-nitrophenylmethyl bromide (4.00 kg) and acetone (15 L). The mixture was stirred until the bromide dissolved (warming may be required). The solution of the bromide was added to the mixture of thiourea, keeping the temperature below 40° C. A thick solution formed within 30 minutes. After stirring for 2 h, HPLC analysis of the mixture shows >99% conversion. The mixture was filtered, and the filter cake was rinsed with a 1:1 MTBE:acetone solution (8 L). The solids were dried to give 5.09 kg (93% yield) of the desired product 6a as a white solid.

### Example 4

### 1-ethyl-3-(5-(5-fluoropyridin-3-yl)-7-(pyrimidin-2-yl)-1H-benzo [d]imidazol-2-yl)urea (1)

NaOAc (1040 g, 12.7 moles, 6.0 equiv.) and water (16 L, 10.5 vol) were charged to a 22 L Morton flask equipped with an overhead stirrer, a pH probe, a thermocouple and two reflux condensers. The pH reading of this solution was 8.3. A concentrated solution of H₂SO₄ (328 mL, 3.94 moles, 0.547 vol) was added until the pH reading of the solution was 3.5. Then 5-(5-fluoropyridin-3-yl)-3-(pyrimidin-2-yl)benzene-1,2-diamine (5) (600 g, 2.13 moles, 1.0 equiv.) was added. The DME solution of 6 (1.2 equiv.) obtained as above in Example 3 was then added directly to the yellow suspension. This heterogeneous mixture was stirred vigorously (200-250 rpm) and heated to reflux (80° C.). During the course of the reaction, the yellow suspension first transformed into a dark yellow suspension, then into a tan suspension. After 4 h, the reaction mixture was cooled to 22˜35° C. The tan suspension was filtered and the filter cake was washed with water (8×2 L), EtOAc (5×4 L), and dried at 40-50° C. under vacuum to provide compound 1 as a beige powder (747 g, 93%, purity: 99.32% AUC by method A). Typical retention times: pseudoisothiourea intermediate (6) 7.3 min, product (1) 6.3 min, SM (5) 5.4 min.

### Example 5

### 1-ethyl-3-(5-(5-fluoropyridin-3-yl)-7-(pyrimidin-2-yl)-1H-benzo [d]imidazol-2-yl)urea, monoesylate salt (monoesylate salt of 1)

1-Ethyl-3-(5-(5-fluoropyridin-3 -yl)-7-(pyrimidin-2-yl)-1H-benzo[d]imidazol-2-yl)urea (1) (51.0 g, 0.135 moles, 1.0 equiv), EtOH (255 mL, 5 vol), ethanesulfonic acid (12.5 mL, 0.149 moles, 1.1 equiv) and H₂O (71.4 mL, 1.4 vol) were charged to a flask under nitrogen equipped with a mechanical stirring apparatus, reflux condenser and a thermometer. The slurry was stirred and heated to 65-70° C. and all solids dissolved, leaving a brown solution, which was cooled to 45-50° C. at a rate of 0.5° C./min and filtered through a 0.2 μm PTFE membrane filter. The filtered reaction mixture was further cooled to 20-25° C. during which time a seed bed was established. After 2 h at 20-25° C., EtOH (255 mL, 5 vol) was added over 30 min and then the mixture cooled to −20˜−15° C. over 2 h and held at −20˜−15° C. for 2 h. The mixture was filtered and the filter cake was washed with cold EtOH (2×5 vol). The filter cake was then transferred to a dish and allowed to dry under vacuum at 40° C. for 12 h to furnish the monoesylate salt of 1 as a light yellow powder (57.6 g, 87.5%, purity: 99.86% AUC by method A.

### Example 5B

### Recovery of the free base from the monoesylate salt of 1

The esylate (1.0 equiv) prepared as indicated above, water (2.5 vol) and 6 N HCl (2.5 vol) were charged to a flask under nitrogen equipped with a mechanical stirring apparatus, reflux condenser and a thermometer. The reaction mixture was stirred for NLT 1 h. The mixture was then filtered through a pad of Celite 454 and activated carbon (1:1 mixture) then a 0.2 μm filter membrane. The filtrate was cooled to 0 to 5° C. at which point 6 N NaOH (˜3.05 vol) was added slowly keeping the temperature below 5° C. until a pH of NLT 11 was reached. The thick white slurry was stirred for NLT 1 h at 0 to 5° C. The slurry was filtered and the filter cake washed with 0.5 N NaOH (2×3 vol), water (2×3 vol) and ethyl acetate (2×10 vol). The filter cake was transferred to a dish and allowed to dry under vacuum at 40-50° C. for 12 h giving the free base as a white powder (60.40 g, 87%)

### Example 6

### 5-fluoropyridine-3-boronic acid (2b)

To a 700 L low temperature reactor were added 3-bromo-5-fluoropyridine (2a) (25 Kg, 142 moles, 1.0 equiv.), THF (222.5 Kg) and isopropyl borate (28 Kg, 149.3 moles, 1.05 equiv.). The resulting mixture was cooled to −90° C.˜−80° C. while stirred. Then n-BuLi (40.2 Kg, 2.5 M, 142 moles, 1.0 equiv.) was added dropwise (2 Kg/h) maintaining the temperature below −87° C. After the addition was complete, the mixture was maintained at −88˜−83° C. for 2.5 h. When the reaction was deemed complete by HPLC analysis, it was quenched by addition of 9% aqueous HCl (7.7 Kg). The mixture was transferred to a 1000 L glass-lined reactor and the temperature returned to −20˜−10° C. Additional HCl solution (122.3 Kg) was then added until pH was adjusted to 1˜2 maintaining the temperature at 0˜10° C. The mixture was then held for 0.5 h in order to allow layers to separate. The organic layer was separated and washed with saturated brine (38 Kg). It was stirred for 0.5 h and then held again for 0.5 h to allow layer separation. The aqueous layer was separated and the combined aqueous layers were extracted with EtOAc twice (51+25 Kg). The organic phase was separated and pH was adjusted to a value of 6 by using 30% aqueous NaOH solution (27.4 Kg). At this pH a solid precipitated out. The slurry was filtered by centrifuge and allowed to dry in a tray dryer at 40˜45° C. Title compound 2b was obtained as a white solid (17.5 Kg, 87.4%, purity: 98.6% AUC using method B).

### Example 7

### 5-fluoropyridine-3-boronic acid pinacolate (2)

To a 1000 L glass-lined reactor was added toluene (20 L/Kg) followed by 5-fluoropyridine-3-boronic acid (2a) (19.45 Kg, 138 moles, 1.0 equiv.) and pinacolate alcohol (16.3 Kg, 138 moles, 1.0 equiv). The resulting mixture was heated to 114˜118° C. and maintained at the same temperature for 21 h. The reaction was monitored by TLC until no SM was detected. Then the mixture was cooled to 80° C. and continued to be cooled to 20˜25° C. At this point it was filtered using a vacuum filter. The filtrate was concentrated under vacuum at T≦80° C. and P<−0.08 MPa until no fraction distilled out. Then, after cooling the mixture to 60° C., cyclohexane (27.4 Kg) was added and the mixture evaporated again under the same conditions until no distillation observed. Maintaining the temperature at 55˜65° C., isopropyl alcohol (20.8 Kg) was added and the resulting mixture was heated to 70˜80° C. and the product was allowed to crystallize at 5˜15° C. for 12 h. The resulting slurry was filtered and the filtration cake was allowed to dry in a tray dryer at 37˜43° C., furnishing the product as a white solid (25 Kg, 81.2%, purity: 98% AUC determined by method C). Typical retention time for product (2) was 9.6 min. ¹H NMR (CDCl₃, 300 MHz): δ 8.75 (1H, s), 8.53 (1H, d), 7.75 (1H, m), 1.96 (12H, s) ppm.

### Example 8

### Preparation of N-(2-bromophenyl)pivalamide (3b)

To a 1000 L glass-lined reactor at 0° C. was added DCM (670 Kg) and Et₃N (76.4 Kg). Then, 2-bromoaniline (3a) (100 Kg, 581 moles, 1.0 equiv.) was added with stirring. The resulting mixture was cooled to 0˜10° C. and pivaloyl chloride (77.1 Kg, 640 moles, 1.1 equiv.) was added dropwise (5 Kg/h) while maintaining the same temperature. The mixture was stirred at this temperature for 1 h 25 min and reaction progress monitored by HPLC. When it was deemed complete, the reaction was quenched by addition of a 5% aqueous solution of Na₂CO₃ (120 Kg) at a rate of 2Kg/min. After addition, the mixture was stirred for 1.5 h and the pH value tested to be between 7 and 8. The reaction was allowed to stand for 20-30 min and the organic phase was separated out. The aqueous layer was extracted with DCM twice (60×2 Kg). During each extraction, the bi-phasic mixture was stirred for 15-20 min and then held for 15˜20° C. to allow for layer separation. All the organic layers were combined and a 3% aqueous solution of HCl (251.8 Kg) was added to adjust the pH value between 5 and 6. Then the aqueous layer was separated and the organic layer was washed with saturated NaHCO₃ (150 L). During the washing, the bi-phasic mixture was stirred for 30 min and held for 30 min. The organic phase was separated and dried over NaSO₄ (50 Kg) with stirring for 13 h. The resulting slurry was filtered under vacuum using a 50 L vacuum filter. The filtration cake was washed with DCM twice (25 Kg×2). The filtrate was concentrated by evaporation at T≦50° C. under atmospheric pressure until no solvent distilled out. Then it was additionally concentrated by evaporation under vacuum (P≦MPa) at 65˜70° C. After concentration, the mixture was cooled to 55˜60° C. and THF was added (993 Kg). Then the mixture was concentrated by evaporation again under the same conditions as above. The content of DCM was monitored by GC using method F until <0.1%. The mixture was cooled to 20˜30° C. under N₂ and then filtered though silica gel (8 Kg) under vacuum in order to dry it. The silica plug was washed twice with THF (20 Kg×2) to give a solution of crude (3b) which was transferred into 200 L drums for the following step. Typical retention time for starting material (3a) is 8.0 min and for product (3b) is 8.9 min.

### Example 9

### N-(2,2-Dimethyl-propionamide)-1-phenyl-boronic acid (3c)

In a 1500 L titanium reactor, under N₂, the above solution of N-(2-bromophenyl)pivalamide (3b) in THF (278 Kg) was mixed with THF (102.7 Kg). With stirring, the resulting solution was cooled to −65˜−70° C. Then n-BuLi (2.5 M, 73.4 Kg, 2.4 equiv.) was added dropwise (2.5/3.0 Kg/h) while keeping the same temperature range. After addition, the reaction was stirred at this temperature for 45 min. The remaining n-BuLi (73.4 Kg) was then added and after the second addition, the mixture was stirred at −60˜−70° C. for 1 h. The reaction was monitored by HPLC until a lithiation ratio of >96% was detected. Then isopropylborate (105.9 Kg, 563 moles, 2.6 equiv.) was added at the rate of 10-15 Kg/h at −60˜−70° C. The resulting mixture was stirred at the same temperature for 4 h 20 min. The reaction was monitored by HPLC until completion. The reaction mixture was quenched by addition of petroleum ether (241.2 Kg) and allowed to warm to 0˜5° C. and maintained at this temperature for 2 h. The resulting mixture was washed-filtered by centrifuge and the cake washed with saturated aqueous NH₄Cl (159 Kg) under stirring for 1 h. Then the washing mixture was also filtered with centrifuge and the combined filtrates were concentrated under vacuum at T≦35° C. and P≦−0.09 MPa until no fraction distilled out. Then the mixture was cooled to 27° C. and centrifuged again. The cake was washed with water (150 Kg) for 0.5-1 h and centrifuged once more. The cake was washed with MTBE (114 Kg) for 0.5-1 h and filtered with centrifugation. After drying the boronic acid (3c) was obtained as an off-white solid (51 Kg, 98.6%, purity: 99.5% using method E). Typical retention time for SM (3b) was 2.3 min.

### Example 10

### N-(2-pyrimidin-2-yl)phenyl)pivalamide (3d)

In a 500 L glass-lined reactor, under N₂, was added glycol dimethyl ether (152.3 Kg, 5 L/Kg). Then 2-chloropyrimidine (18.4 Kg, 160 moles, 1.0 1 equiv.) was added with stirring, followed by Pd[(Ph₃)P]₄ (3.65 Kg, 3.2 moles, 0.02 equiv.). The resulting mixture was stirred at 15˜25° C. for 20-30 min and then N-(2,2-dimethyl-1-propinamide)1-phenyl-boronic acid (3c) (35 Kg of product, containing additional 11 Kg of salts, 158 moles, 1.0 equiv.) was added in one portion. After addition, aqueous Na₂CO₃ solution (132.8 Kg, 2 M) was added quickly and the mixture heated to 78˜83° C. and refluxed for 3 h. The reaction was monitored by HPLC using method H until SM (3c) was <3%. The mixture was then slightly cooled to 70˜75° C., quenched by addition of cold purified water (5˜10° C., 525 Kg) and stirring continued at 0˜10° C. for 1-2 h. The mixture was then filtered by centrifuge and the cake washed with purified water. The wet product from 4 batches (7, 35, and 35 Kg scale) was combined and dried on a tray dryer for a week and then on a rotary conical dryer but some water (at least 3% weight) could not be removed. The title compound (3d) was obtained as a yellow wet solid (157 Kg, >100%, purity>98% determined by method F). Typical retention time for product was 15.9 min.

### Example 11

### Preparation of N-(4-Bromo-2-(pyrimidin-2-yl)pivalamide (3e)

Acetic acid (367.5 Kg) was charged to a 500 L reactor. Then, N-(2-(pyrimidin-2-yl)phenyl)pivalamide (3d) (35 Kg of product, contained water, 137.3 moles, 1.0 equiv.) was added followed by NBS (26.9 Kg, 151.1 moles, 1.1 equiv.). The resulting mixture was heated to 50˜55° C. and stirred for 5 h. The reaction progress was monitored by HPLC (method I) until 3d was <2%. The reaction was quenched by pouring onto purified water (350 Kg) which had previously been cooled to 0˜5° C. The mixture was maintained at 0˜10° C. under stirring for 1-2 h. The mixture was then filtered via a centrifuge. The combined cake from 3 batches (from 35 Kg of 3d each) was washed with purified water twice (550+500 Kg) under stirring for 0.5-1 h. After a second filtration with centrifuge and drying, the title product (3e) was obtained as a light yellow solid (105.6 Kg, 80%, purity: 99.3% determined by analytical method G). Typical retention time for SM (3d) was 7.5 min, for product (3e) was 15.1 min.

### Example 12

### Preparation of N-(4-bromo-2-nitro-6-pyrimidin-2-yl)phenyl)pivalamide (3f)

Water (7.4 Kg) was charged to a 200 L reactor. Upon cooling to 0˜5° C., conc. H₂SO₄ (138 Kg, 98%, 3.77 L/Kg) was added at a temperature T≦30° C. The resulting mixture was cooled to 0˜5° C. again and N-(4-bromo-2-pyrimidin-2-yl)phenyl)pivalamide (3e) (20 Kg, 59.8 moles, 1.0 equiv.) was added in 4 portions keeping the temperature constant. After addition, the mixture was stirred at 5˜10° C. for 0.5-1 h until the solid dissolved completely. After cooling to −10˜−5° C., HNO₃ (12 Kg, 98%, 0.4 L/Kg) was added dropwise (2 Kg/h) maintaining this temperature. After addition the mixture was stirred at the same temp. for 30 min. The reaction progress was monitored by TLC until no SM (3e) could be detected. While maintaining the temperature at −10˜−0° C. the reaction was quenched by pouring it onto a mixture of crushed ice (280 Kg) and tap water (420 Kg) in a 1000 L glassed-lined reactor. The mixture was then maintained at 0˜10° C. for 0.5-1 h. The mixture was filtered with centrifuge and the cake was washed until its pH was between 6 and 7. The resulting product was dried at 50˜60° C. The crude products from several batches (3×20 Kg scale) were combined and dissolved in DCM (469 Kg), dried over Na₂SO₄ (25 Kg) under stirring for 4 h and press-filtered through silica gel (40 Kg). The silica plug was washed with DCM twice (90 Kg×2) and the combined filtrates were concentrated under vacuum at T<50° C. to remove the solvent. Petroleum ether was then added (141.7 Kg) and the solution concentrated again at 40˜50° C. until no fraction distilled out. The resulting crude was filtered via a centrifuge. The cakes were combined furnishing the title compound (3f) as a grayish brown solid (62 Kg, 91%, purity: >92.7% as determined by method G). Typical retention time for product (3f) was 11.3 min.

### Example 13

### Preparation of 4-bromo-2-nitro-6-pyrimidin-2-yl-phenylamine (3)

In a 500 L was charged EtOH (87.4 Kg, 3.12 Kg/Kg). With stirring, a solution of HCl in EtOH (168 Kg, 35%, 6 Kg/6 Kg) was then added. Finally, N-(4-bromo-2-nitro-6-pyrimidin-2-yl)phenyl)pivalamide (3f) (28 Kg, 73.8 moles, 1.0 equiv.) was added in one portion. After addition, the mixture was heated to 80˜88° C. and refluxed for 49 h. The reaction progress was monitored by HPLC (method I) until 3f was ≦3%. Additional HCl/EtOH solution (50.5 Kg) was added and the mixture continued to be refluxed for an additional 9.5 h. The mixture was cooled to 30˜40° C. and quenched by pouring it onto cold purified water (280 Kg). The mixture was filtered with centrifuge and the cake was washed with water twice under stirring for 1 h (450 Kg×2). The resulting solid was dried at 40˜50° C. under N₂, to furnish the title compound 3 as a brownish yellow solid (20.9 Kg, 96%, purity: 99.0% determined by method G). Typical retention time for 3 was 10.1 min. ¹HNMR(CDC1₃, 300 MHz): δ 9.00 (1H, d), 8.85 (1H, d), 8.46 (1H, d), 7.29 (2H, m) ppm.

1. A process for preparing 1 -ethyl-3 -(5 -(5 -fluoropyridin-3 -yl)-7-(pyrimidin-2-yl)-1H-benzo[d]imidazol-2-yl)urea of formula 1: 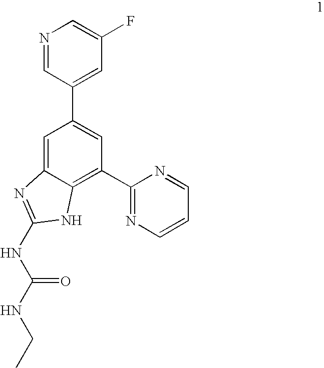
comprising:
i) providing 3-fluoro-5-(4,4,5,5-tetramethyl-1,3,2-dioxaborolan-2-yl)pyridine (formula 2) and 4-bromo-2-nitro-6-(pyrimidin-2-yl)benzenamine (formula 3), 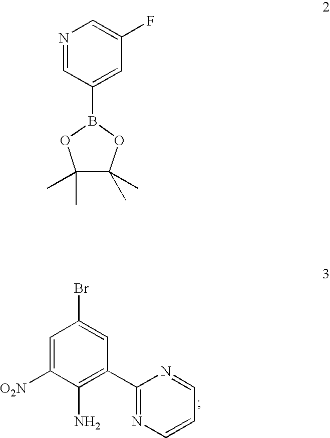
ii) cross coupling the compound of formula 2 and the compound of formula 3 in a biphasic mixture comprising water, an organic solvent, a base and a transition-metal catalyst to produce 4-(5-fluoropyridin-3-yl)-2-nitro-6-(pyrimidin-2-yl)benzenamine of formula 4, 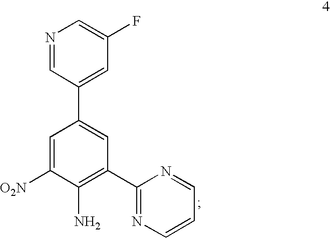
iii) reducing the compound of formula 4 to produce 5-(5-fluoropyridin-3-yl)-3-(pyrimidin-2-yl)benzene-1,2-diamine of formula 5, 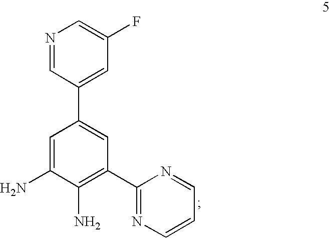
iv) reacting the compound of formula 5 with the compound of formula 6, 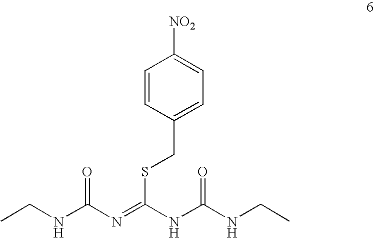
in a biphasic mixture comprising buffered water and an organic solvent to produce the compound of formula 1.

2. The process of claim 1, wherein the organic solvent in ii) is an aprotic solvent.

3. (canceled)

4. The process of claim 1, wherein the base in ii) is an inorganic base.

5. (canceled)

6. The process of claim 1, wherein the transition-metal catalyst in ii) is a palladium-based catalyst.

7. (canceled)

8. The process claim 1, wherein said biphasic mixture in ii) additionally comprises a phosphine ligand.

9-10. (canceled)

11. The process of claim 1, wherein said biphasic mixture in ii) additionally comprises a phase transfer catalyst.

12. (canceled)

13. The process of claim 1, wherein the cross coupling reaction of ii) is run at between 75° C. to 120° C.

14-15. (canceled)

16. The process of claim 1, wherein in iii) the compound of formula 4 is reduced under catalytic hydrogenation conditions comprising a suitable hydrogen atmosphere and a suitable organic solvent.

17-19. (canceled)

20. The process of claim 1, wherein the organic solvent in iv) is an aprotic solvent.

21-24. (canceled)

25. The process of claim 20, wherein the biphasic buffered water solution in iv) comprises an aprotic solvent and an aqueous buffer adjusted to a pH of between 2 to 5.

26-27. (canceled)

28. The process of claim 1, wherein the reaction of iv) is run at between 50° C. to 100° C.

29-30. (canceled)

31. The process of claim 1, further comprising:
a) slurrying a preparation comprising the compound of formula 1 in an organic solvent, water and acid to obtain a suspension of the compound of formula 1,
b) heating the suspension to obtain a homogeneous solution of compound 1,
c) filtering the homogeneous solution of compound 1,
d) cooling the solution to obtain a salt of compound 1 in a solid form.

32. The process of claim 31, wherein in a) and b) said organic solvent is a protic solvent, said acid is a sulfonic acid and said heating is to a temperature of between 40° C. to 90° C.

33-34. (canceled)

35. The process of claim 31, further comprising:
e) recrystallizing the salt of compound 1 in a suitable organic solvent or a mixture of an organic solvent and water at a suitable temperature.

36-37. (canceled)

38. A process for purifying a compound of formula 1, 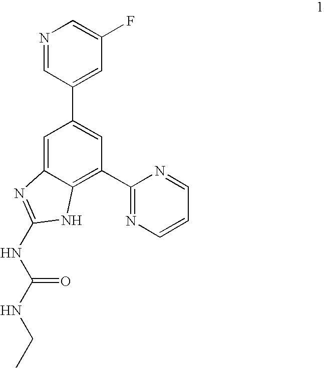
comprising:
a) slurrying a preparation comprising a compound of formula 1 in an organic solvent, water and acid to obtain a suspension of the compound of formula 1,
b) heating the suspension to obtain a homogeneous solution of compound 1,
c) filtering the homogeneous solution of compound 1,
d) cooling the solution to obtain a salt of compound 1 in a solid form.

39. The process of claim 38, wherein said organic solvent is a protic solvent, said acid is a sulfonic acid and said heating is to a temperature of between 40° C. to 90° C.

40-41. (canceled)

42. The process of claim 38, further comprising recrystallizing the salt of compound 1 in a suitable organic solvent or a mixture of an organic solvent and water at a suitable temperature.

43-44. (canceled)

45. A process for preparing a compound of formula 1: 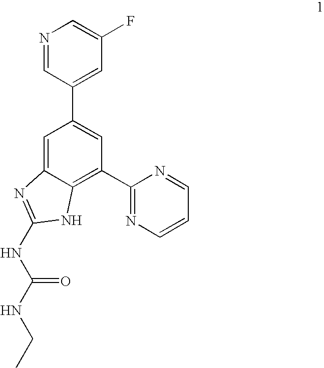
comprising:
ia) reacting 5-(5-fluoropyridin-3-yl)-3-(pyrimidin-2-yl)benzene-1,2-diamine of formula 5, 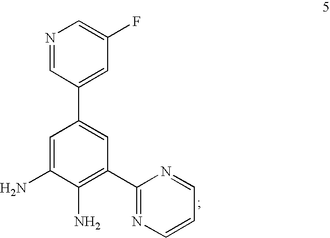
with a compound of formula 6, 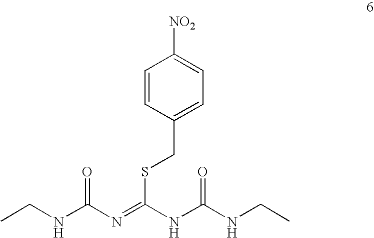
in a biphasic mixture comprising buffered water and an organic solvent to produce the compound of formula 1.

46. A process for preparing a compound of formula I: 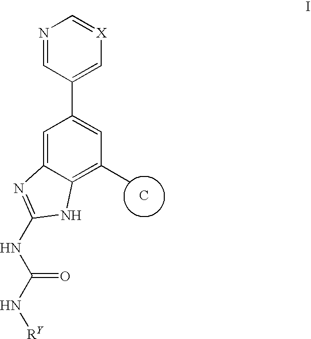
comprising:
ib) reacting a compound of formula 4b, 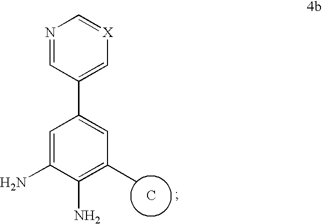
wherein,
Ring C is a 6-membered heteroaryl ring having 1-2 nitrogens, wherein:
Ring C is substituted with 1-3 R¹ groups;
each R¹ is independently selected from OR² or halogen; and
R² is C₁₋₄ aliphatic; or
Ring C is an unsubstituted 2-pyrimidine ring;
X is nitrogen, CH, or CF;:
with a compound of formula 6b, 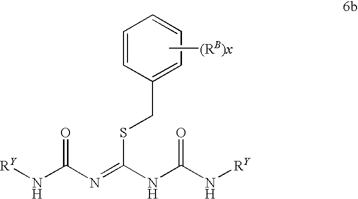
wherein,
x is 0-5;
R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}(O)R^{J}, —SOR^{J}, —SO₂R^{J}, —SO₂N(R^{J})₂, —SO₂N(R^{J})₂, —NR^{J}SO₂R^{J}, —COR^{J}, —NR^{J}SO₂N(R^{J})₂, —COCOR^{J}; wherein
R^{J} is hydrogen or unsubstituted C₁₋₆ aliphatic; and
each R^{Y} is C₁₋₆ aliphatic;
in a biphasic mixture comprising buffered water and an organic solvent to produce the compound of formula I.

47. (canceled)

48. A compound of formula 6b: 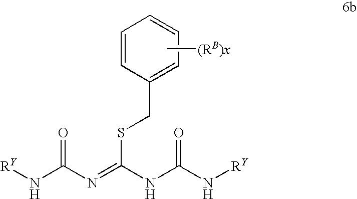
wherein
x is 0-5;
R^{B} is selected from —R^{J}, —OR^{J}, —N(R^{J})₂, —NO₂, halogen, —CN, —C₁₋₄ haloalkyl, —C₁₋₄ haloalkoxy, —C(O)N(R^{J})₂, —NR^{J}C(O)R^{J}, —SOR^{J}, —SO₂R^{J}, —SO₂N(R^{J})₂, —NR^{J}SO₂R^{J}, —COR^{J}, —NR^{J}SO₂N(R^{J})₂, —COCOR^{J}; wherein
R^{J} is hydrogen or unsubstituted C₁₋₆ aliphatic; and
each R^{Y} is C₁₋₆ aliphatic.

49-50. (canceled)

51. A compound of formula 4a: 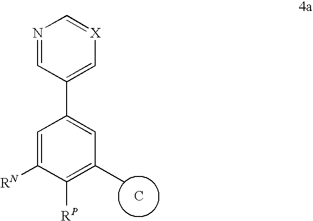
wherein
Ring C is a 6-membered heteroaryl ring having 1-2 nitrogens, wherein:
Ring C is substituted with 1-3 R¹ groups;
each R¹ is independently selected from OR² or halogen; and
R² is C₁₋₄ aliphatic; or
Ring C is an unsubstituted 2-pyrimidine ring;
X is nitrogen, CH, or CF;
R^{N} and R^{P} are independently NO₂, NH₂ or NHR^{W};
R^{W} is an amino protecting group;
provided that the following compounds are excluded:
2-nitro-6-(pyridin-2-yl)-4-(pyridin-3-yl)phenyl)amine; 3-(pyridin-2-yl)-5-(pyridin-3-yl)benzene-1,2-diamine; and (2-nitro-3-(pyrazol-1-yl)-5-(pyridin-3-yl)phenyl)amine.

52-54. (canceled)
